(19) United States
(12) Patent Application Publication
(10) Pub. No.: US 2025/0302563 A1
(43) Pub. Date: Oct. 2, 2025

(54) MITIGATING MISMATCHED INPUT DEVICE DURING TELEOPERATION

(71) Applicant: Intuitive Surgical Operations, Inc., Sunnyvale, CA (US)

(72) Inventors: Kevin Supakkul, Mountain View, CA (US); Joseph Giallo, Cary, NC (US); Junjie Zhang, San Jose, CA (US); Kyle Petrich, Los Gatos, CA (US)

(73) Assignee: Intuitive Surgical Operations, Inc., Sunnyvale, CA (US)

(21) Appl. No.: 19/093,722

(22) Filed: Mar. 28, 2025

Related U.S. Application Data
(60) Provisional application No. 63/571,735, filed on Mar. 29, 2024.
(60) Provisional application No. 63/571,756, filed on Mar. 29, 2024.

(51) Int. Cl. A61B 34/00; A61B 34/20; A61B 34/35

(52) U.S. Cl. CPC A61B 34/70; A61B 34/20; A61B 34/35; A61B 2034/2055

(57) ABSTRACT

A computer-assisted system includes: a first input device configured to be manipulated by an operator; a manipulator assembly configured to support an imaging device and a first instrument (the imaging device has a field of view associated with a view coordinate frame); and a control system communicatively coupled to the first input device and the manipulator assembly. The control system is configured to: perform a first association between the first input device and the first instrument; determine a first instrument vector representative of an orientation of a shaft of the first instrument; perform a first comparison between the first instrument vector and a first view frame vector, the first comparison being performed based on the first association; based on the first comparison, determine whether a mismatch condition is satisfied; and, in response to determining that the mismatch condition is satisfied, perform a mitigation action.

### CROSS-REFERENCE TO RELATED APPLICATIONS

This Patent Application claims the benefit of priority under 35 U S.C. § 119(e) to U.S. Provisional Patent Application Ser. No. 63/571,735, filed on Mar. 29, 2024, which is hereby incorporated by reference herein in its entirety. This Patent Application further claims the benefit of priority under 35 U S.C. § 119(e) to U.S. Provisional Patent Application Ser. No. 63/571,756, filed on Mar. 29, 2024, which is hereby incorporated by reference herein in its entirety.

### BACKGROUND

### Field of Invention

The present invention generally provides improved computer-assisted devices, systems, and methods.

### Overview

Computer-assisted systems can be used to perform a task at a workspace. For example, a computer-assisted system may comprise an input system (e.g., a console, a surgeon's console), a manipulator assembly, and a control system. For example, in a medical context, the manipulator assembly may be used to perform diagnosis, non-surgical treatment, surgical treatment (e.g., through minimally invasive apertures or natural orifices) and is guided by the user based on images or video from the imaging device.

As another example, a computer-assisted system may comprise a robotic system (e.g., industrial and recreational systems), and may include one or more robotic manipulators to manipulate instruments for performing the task.

The computer-assisted system can be automated, semi-automated, teleoperated, or any combination thereof. In any mode of operation, a high degree of coordination of the manipulator assembly is required. If coordination of the manipulator assembly is lost or degraded, operation of the computer-assisted system may result in unintended movements. Therefore, an efficient, reliable, and/or easier-to-perform method of maintaining/evaluating coordination of the computer-assisted systems is, therefore, highly desirable.

### SUMMARY

In general, in one aspect, one or more embodiments relate to a computer-assisted system including: a first input device configured to be manipulated by an operator; a manipulator assembly configured to support an imaging device and a first instrument (the imaging device has a field of view associated with a view coordinate frame); and a control system communicatively coupled to the first input device and the manipulator assembly. The control system is configured to: perform a first association between the first input device and the first instrument; determine a first instrument vector representative of an orientation of a shaft of the first instrument; perform a first comparison between the first instrument vector and a first view frame vector, the first comparison being performed based on the first association of the first input device with the first instrument; based on the first comparison, determine whether a mismatch condition is satisfied;

and in response to determining that the mismatch condition is satisfied, perform a mitigation action.

In general, in one aspect, one or more embodiments relate to method of operating a computer-assisted system including a first input device configured to be manipulated by an operator, a manipulator assembly configured to support an imaging device and a first instrument, and a control system. The method comprising: performing, by the control system, a first association between the first input device and the first instrument; determining, by the control system, a first instrument vector representative of an orientation of a shaft of the first instrument in a view coordinate frame associated with a field of view of the imaging device; performing, by the control system, a first comparison between the first instrument vector and a first view frame vector, the first comparison being performed based on the first association of the first input device with the first instrument; determining, by the control system and based on the first comparison, whether a mismatch condition is satisfied; and in response to determining that the mismatch condition is satisfied, perform a mitigation action.

Other aspects of the invention will be apparent from the following description and the appended claims.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a simplified diagram and schematic of an example computer-assisted system, in accordance with one or more embodiments.

FIG. 2A shows a user interface system, in accordance with one or more embodiments.

FIG. 2B is a perspective view of a controller portion 200 of an example input device 152 of the user interface system 120 shown in FIG. 2A.

FIGS. 2C-2D show examples of a manipulator assembly, in accordance with one or more embodiments.

FIG. 3 is a simplified diagram showing example coordinate frames relevant to a computer-assisted system 100, in accordance with one or more embodiments.

FIGS. 4A-4B show example fields of view 322 of an imaging device 320, in accordance with one or more embodiments.

FIGS. 5A-5B show a method according to one or more embodiments.

FIGS. 6A-6B show a method according to one or more embodiments.

FIG. 7 shows a method according to one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements, and is not to limit any clement to being only a single clement unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

This disclosure describes various devices, elements, and portions of computer-assisted systems and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to computer-assisted systems, which can include devices that are teleoperated, externally manipulated, autonomous, semiautonomous, and/or the like. Further, aspects of this disclosure are described in terms of an implementation using a teleoperated surgical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperated systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

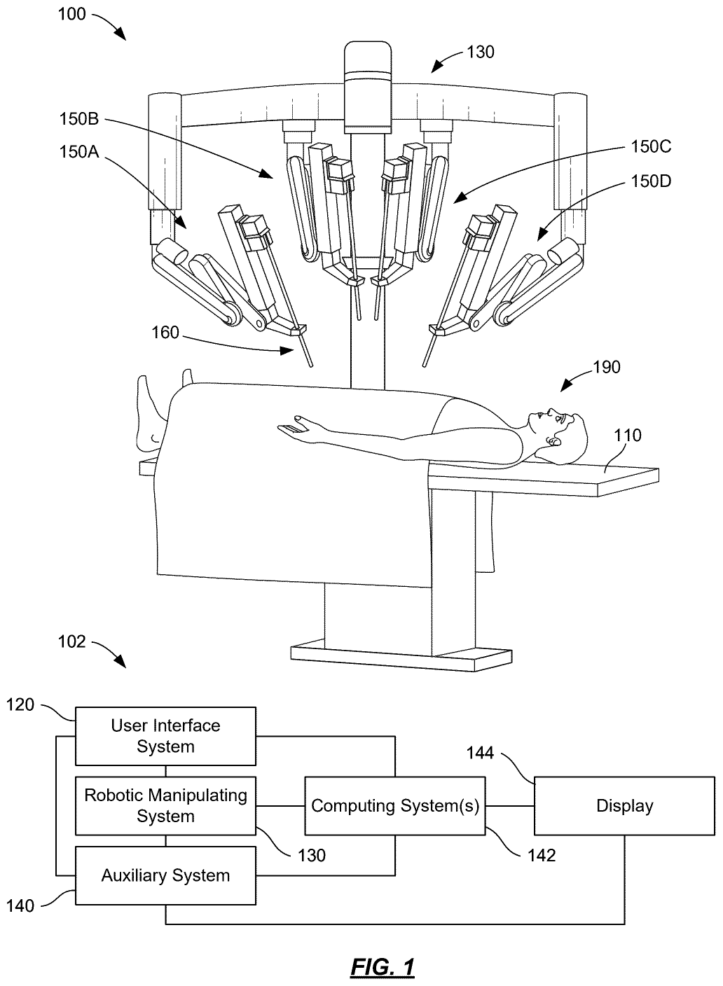

FIG. 1 shows an example computer-assisted system 100, in accordance with one or more embodiments.

In the example, a diagnostic or therapeutic medical procedure is performed on a patient 190 on an operating table 110. The computer-assisted system 100 may include a manipulator assembly 130 (e.g., a patient-side robotic device in a medical example). The manipulator assembly 130 may include at least one manipulator arm 150 (e.g., a robotic manipulator arm). A manipulator arm 150 may be any type of manipulator (e.g., a general-purpose robotic arm, a robotic arm designed for a specific application (e.g., a medical device robotic arm)). A manipulator arm 150 may include any number of links that are coupled by joints of any type (e.g., revolute joints, prismatic joints). In some embodiments, multiple manipulator arms 150 may be modular components of a manipulator assembly 130 (e.g., multiple stations or carts that each support an independent manipulator arm 150) or part of an integrated manipulator assembly 130 (e.g., a multi-arm station or cart that supports multiple manipulator arms 150).

One or more of the manipulator arms 150 may support a removably coupled instrument 160 (also called tool 160). In the manipulator assembly 130 and manipulator arm(s) 150 may maneuver the instrument 160 to a workspace through an entry location (e.g., enter the body of the patient 190 through a natural orifice such as the throat or anus, or through an incision), while an operator (not shown) views the workspace (e.g., a surgical site in the surgical scenario) through a user interface system 120.

An image of the workspace may be obtained by an instrument 160 comprising an imaging device (e.g., an endoscope, an optical camera, an ultrasonic probe, etc. in a medical example). The imaging device can be used for imaging the workspace, and may be manipulated by one of the manipulator arms 150A-D of the manipulator assembly 130 so as to position and orient the imaging device. The auxiliary system 140 may process the captured images in a variety of ways prior to any subsequent display. For example, the auxiliary system 140 may overlay the captured images with a virtual control interface prior to displaying the combined images to the operator via the user interface system 120 or other display systems located locally or remotely from the procedure. One or more separate displays 144 may also be coupled with a control system 142 and/or the auxiliary system 140 for local and/or remote display of images, such as images of the procedure site, or other related images.

The number of instruments 160 used at one time generally depends on the task and space constraints, among other factors. If it is appropriate to change, clean, inspect, or reload one or more of the instruments 160 being used during a procedure, an assistant (not shown) may remove the instrument 160 from a manipulator arm 150, and replace it with the same instrument 160 or another instrument 160.

The computer-assisted system 100 may include a control system 142 (e.g., a computing system). The control system 142 may be used to process input provided by the user interface system 120 from an operator, such as to control the computer-assisted system 100. The control system 142 may also be used to process signals from other devices, from sensors, from any networks to which the control system 142 connects, etc. Example sensors include those associated with actuators or joints of the computer-assisted system, such as motor encoders, rotary or linear joint encoders, torque sensors, current sensors, accelerometers, force sensors, inertial measurement units, optical or ultrasonic sensors or imagers, RF sensors, etc. The control system 142 may further be used to provide an output (e.g., a video image for display by the display 144). The control system 142 may further be used to control the robotic manipulator assembly 130.

The control system 142 may include one or more computer processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities.

A computer processor of the control system 142 may be part or all of an integrated circuit for processing instructions. For example, the computer processor may be one or more cores or micro-cores of a processor. The control system 142 may also communicate with one or more input devices, such as a touchscreen, keyboard, mouse, microphone, touchpad, electronic pen, or any other type of input device.

A communication interface of the control system 142 may include an integrated circuit for connecting the control system 142 to a network (not shown) (e.g., a local area network (LAN), a wide area network (WAN) such as the Internet, mobile network, or any other type of network) and/or to another device, such as another control system 142.

Further, the control system 142 may communicate with one or more output devices, such as a display device (e.g., a liquid crystal display (LCD), a plasma display, touchscreen, organic LED display (OLED), projector, or other display device), a printer, a speaker, external storage, or any other output device. One or more of the output devices may be the same or different from the input device(s). Many different types of control systems exist, and the aforementioned input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

A control system 142 may be connected to or be a part of a network. The network may include multiple nodes. Each node may correspond to a computing system, or a group of nodes. By way of an example, embodiments of the disclosure may be implemented on a node of a distributed system that is connected to other nodes. By way of another example, embodiments of the invention may be implemented on a distributed computing system having multiple nodes, where each portion of the disclosure may be located on a different node within the distributed computing system. Further, one or more elements of the aforementioned computing system may be located at a remote location and connected to the other elements over a network.

While FIG. 1 shows the computer-assisted system 100 as a medical system, the following description is applicable to other scenarios and systems (e.g., medical scenarios or systems that are non-surgical, non-medical scenarios or computer-assisted systems, etc.).

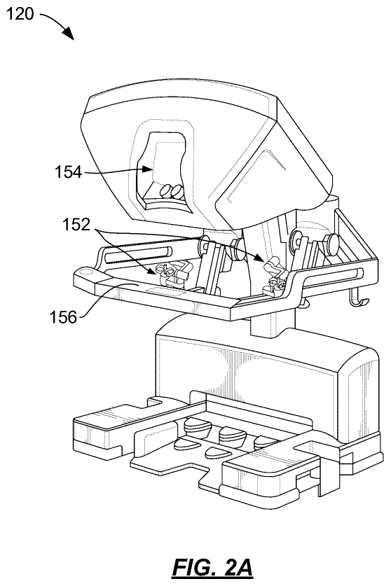

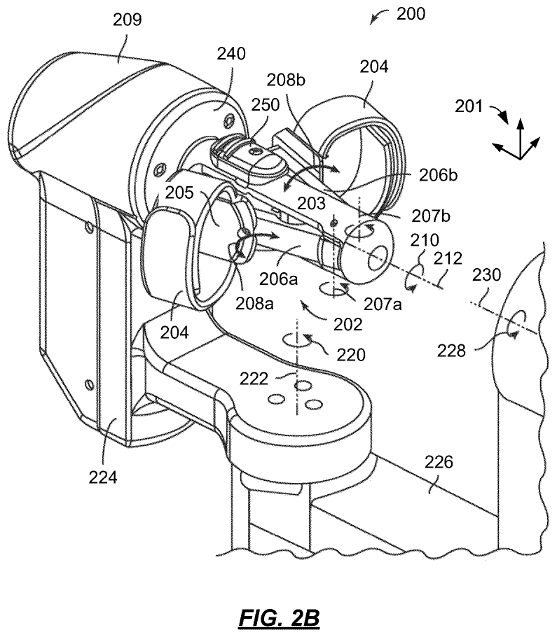

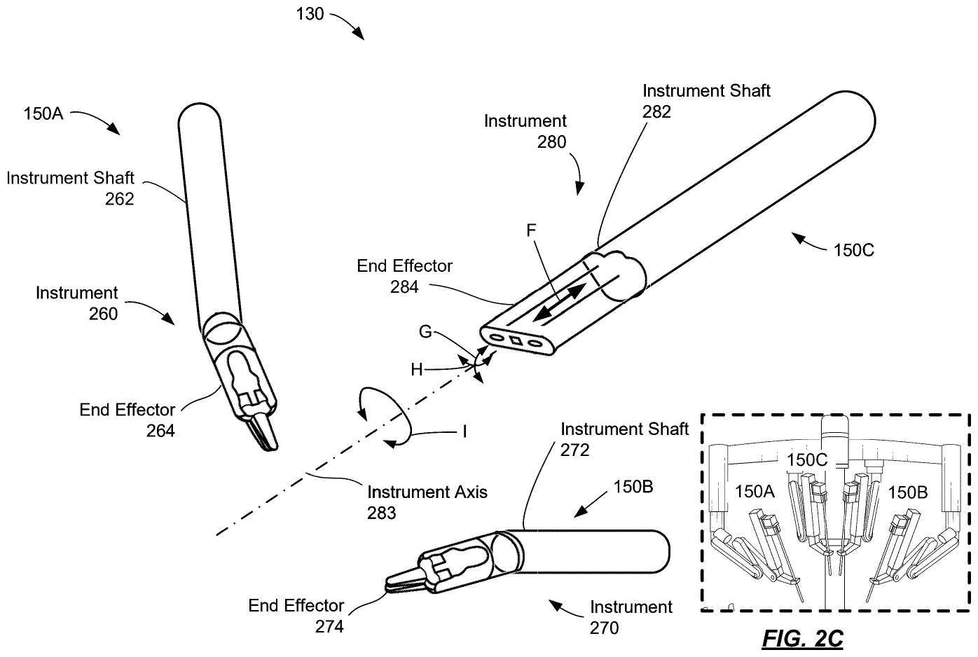

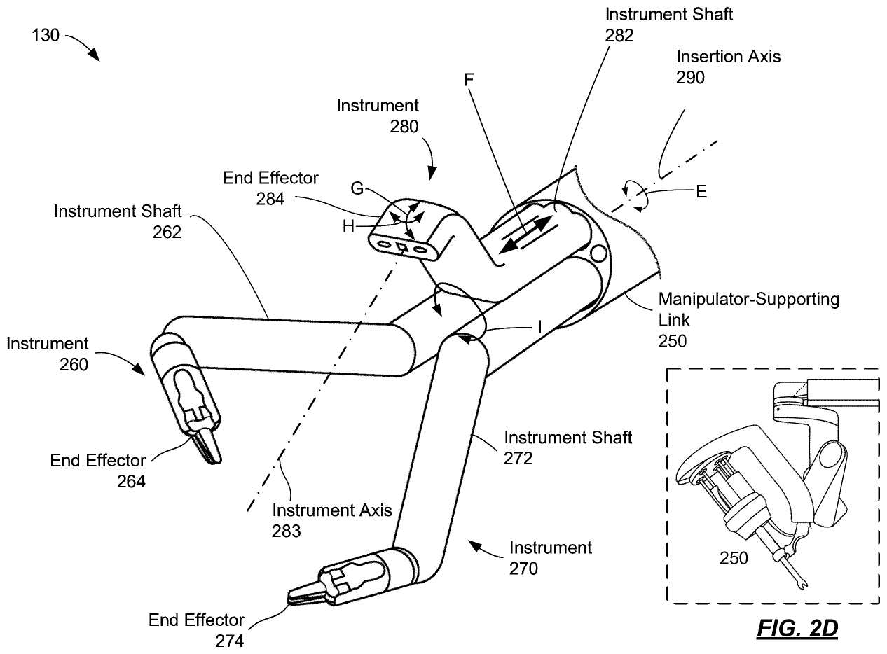

FIG. 2A shows a user interface system, in accordance with one or more embodiments.

In some embodiments, the user interface system 120 includes one or more input devices 152 operated by the operator (not shown). The one or more input devices 152 are contacted and manipulated by the hands of the operator, with one input device for each hand.

Examples of such hand-input-devices include any type of device manually operable by human operator (e.g., joysticks, trackballs, button clusters, and/or other types of haptic devices typically equipped with multiple degrees of freedom). A more detailed description of an input device 152 is provided below in reference to FIG. 2B. Additionally, in some embodiments, position, force, and/or tactile feedback devices (not shown) are employed to transmit position, force, and/or tactile sensations from the instruments back to the operator's hands through the input devices 152.

The input devices 152 are supported by user interface system 120 and are shown as mechanically grounded, and in other implementations may be mechanically ungrounded. An ergonomic support 156 is provided in some implementations. For example, FIG. 2A shows an ergonomic support 156 including forearm rests on which the operator may rest his or her forearms while manipulating the input devices 152. In some examples, the operator performs tasks at a work site near the manipulator assembly 130 during a medical procedure by controlling the manipulator assembly 130 using the input devices 152.

A display unit 154 is included in the user interface system 120. The display unit 154 displays images for viewing by the operator. The display unit 154 provides the operator with a view of the workspace with which the manipulator assembly 130 interacts. The view can include, for example, stereoscopic images or three-dimensional images to provide a depth perception of the workspace and the instrument(s) 160 of the manipulator assembly 130 in the workspace. The display unit 154 can be moved in various degrees of freedom to accommodate the operator's viewing position and/or to provide control functions. Where a display unit (such as the display unit 154 is also used to provide control functions, such as to command the manipulator assembly, the display unit also includes an input device (e.g., another input device 152).

When using user interface system 120, the operator can sit in a chair or other support in front of user interface system 120, position his or her eyes to see images displayed by the display unit 154, grasp and manipulate the input devices 152, and rest his or her forearms on the ergonomic support 156 as desired. In some implementations, the operator can stand at the workstation or assume other poses, and the display unit 154 and input devices 152 may differ in construction and/or be adjusted in position (e.g., height, depth, etc.).

FIG. 2B is a perspective view of a controller portion 200 of an example input device 152 of the user interface system 120 shown in FIG. 2A.

In some implementations, the controller portion 200 includes one or more gimbal mechanisms. In the example of FIG. 2B, the controller portion 200 includes a handle 202 which is contacted by an operator to manipulate the input device. Further, in this example, the handle 202 includes two grips that each include a finger loop 204 and a grip member (depicted as grip members 206a, 206b). The two grip members 206a,b are positioned on opposite sides of a central portion 203 of the handle 202, and the grip members 206a,b can be grasped, held, or otherwise contacted by an operator's fingers. Each finger loop 204 is attached to a respective grip member 206a or 206b and can be used to secure an operator's fingers to the associated grip member 206a or 206b. In this example, finger contacts 205 can be connected or formed at the unconnected end of the grip members 206a,b to provide surfaces to contact the operator's fingers. The operator may also contact other portions of handle 202 while grasping the grip members 206a,b.

Each grip member 206a,b and finger loop 204 can be moved in an associated degree of freedom (depicted as degrees of freedom 208a,b). In some examples, the grip members 206a,b are each coupled to the central portion 203 of the handle 202 at respective rotational couplings, allowing rotational movement of the grip members 206a,b about associated grip axes 207a,b, with respect to the central portion 203. As such, each grip member 206 can be moved in an associated degree of freedom (i.e., degree of freedom 208a about grip axis 207a and degree of freedom 208b about grip axis 207b) by an operator contacting the grip members 206a,b. In various implementations, a single grip member 206 (e.g., 206a) and finger loop 204 can be provided, or only one of the grip members 206 can be moved in the corresponding degree of freedom 208 while the other grip member (e.g., 206b) can be fixed with reference to the handle 202. For example, the positions of grip members 206a,b in their degrees of freedom 208a,b can control corresponding rotational positions of an instrument 160 or component thereof.

One or more grip sensors (not shown) can be coupled to the handle 202 and/or other components of the controller portion 200 and can detect the positions of the grip members 206a,b in their respective degrees of freedom 208a,b. The grip sensors can send signals describing sensed positions and/or motions to the control of control system 142 of the computer-assisted system 100. In some modes or implementations, the control system 142 can provide control signals to a device manipulated by the computer-assisted system 100 (e.g., manipulator assembly 130). For example, the positions of the grip members 206a,b in their respective degrees of freedom 208a,b can be used to control any of various degrees of freedom of an instrument 160 (or, in some instances, the distal end of an instrument 160) supported by the manipulator assembly 130.

Various implementations of an input device, such as the that depicted in the controller portion 200 of FIG. 2B, can provide one or more active actuators (e.g., motors, voice coils, etc.) to output active forces on the grip members 206a,b in the degrees of freedom 208a,b. For example, a sensor and/or actuator can be housed in central portion 203 or in housing 209 and coupled to the grip members 206a,b by a transmission. Some implementations can provide one or more passive actuators (e.g., brakes) or springs between the grip members 206a,b and the central portion 203 of the handle 202 to provide resistance in particular directions of the grips (e.g., movement in directions toward each other in degrees of freedom 208a,b).

The handle 202 can additionally be provided with a rotational degree of freedom 210 about a roll axis 212 defined between a first end and a second end of the handle 202. The roll axis 212 is a longitudinal axis in this example that extends approximately along the center of the central portion 203 of handle 202. The handle 202 can be rotated about the roll axis 212 with respect to a base member of the controller portion 200, such as a base member that includes the housing 209. For example, an operator can rotate the grip members 206a,b and central portion 203 as a single unit around the roll axis 212), with respect to the housing 209, to provide control of manipulator assembly 130 or other elements.

Additionally, one or more input sensors (not shown) can be coupled to the handle 202 to detect the orientation of the handle 202 in the rotational degree of freedom 210. For example, the sensor can send signals describing the orientation to the control system 142 that can provide control signals to the manipulator assembly 130 as described above. For example, rotation of the handle 202 in the rotational degree of freedom 210 can control a particular degree of freedom of an instrument 260, 270, 280 of the manipulator assembly 130 that is different than another degree of freedom controlled by the degrees of freedom 208a,b of the grip members 206a,b.

Some implementations of the controller portion 200 can provide one or more actuators to output forces on the handle 202 (including grip members 206a,b and finger loops 204 in the rotational degree of freedom 210. For example, a sensor and/or actuator can be housed in the housing 209 and coupled to the handle 202 by a shaft extending through the central portion 203 of the handle 202.

In various implementations, the handle 202 can be provided with additional degrees of freedom. For example, a rotational degree of freedom 220 about a yaw axis 222 can be provided to the handle 202 at a rotational coupling between an elbow shaped link 224 and a link 226, where the elbow shaped link 224 is coupled to the handle 202 (e.g., at the housing 209). In this example, the yaw axis 222 intersects and is orthogonal to the roll axis 212. Additional degrees of freedom can similarly be provided. For example, the link 226 can be elbow-shaped and a rotational coupling can be provided between the other end of link 226 and another link (not shown). A rotational degree of freedom 228 about an axis 230 can be provided to the handle 202 at the rotational coupling. In some examples, the controller portion 200 can allow movement of the handle 202 within the workspace of the user interface system 120 with a plurality of degrees of freedom (e.g., six degrees of freedom including three rotational degrees of freedom and three translational degrees of freedom). One or more additional degrees of freedom can be sensed by associated input sensors and/or actuated by actuators (e.g., motors, etc.), similarly as described above for the degrees of freedom, coupled to the controller portion 200. In various implementations, sensors can sense positions of the handle in a degree of freedom, or sense orientations of the handle in a degree of freedom, or sense positions and orientations of the handle in multiple degrees of freedom. For example, positions in a translational degree of freedom and orientations in a rotational degree of freedom can be sensed by one or more associated input sensors. In some examples, a position in a translational degree of freedom and/or orientation in a rotational degree of freedom can be derived from rotations of components (e.g., links of a linkage) coupled to the handle 202 as sensed by rotational sensors. Some implementations can include linear sensors that can directly sense translational motion of one or more components coupled to the handle 202. In some implementations, each additional degree of freedom of the handle 202 can control a different degree of freedom (or other motion) in the manipulator assembly 130.

In an example implementation, the handle 202 is mechanically grounded, i.e., supported in space by a kinematic chain with an end stationary at mechanical ground, such as a floor, wall, or ceiling. For example, the housing 209 can be coupled to a mechanical linkage that is coupled to the ground or an object connected to ground, providing a stable platform for the use of the controller portion 200. For example, a grounded mechanical linkage can be connected to a base member (e.g., with one or more rotary couplings, ball joints, or other couplings, including linear joints). The mechanical linkage can provide six or more degrees of freedom to the handle 202.

In the example of FIG. 2B, the handle 202 includes one or more control switches 240. As depicted, the one or more control switches 240 can be coupled to the central portion 203 or to mechanisms within central portion 203. For example, two control switches 240 can be positioned on opposite sides of axis 212, and/or additional control switches can be provided. In some examples, a control switch 240 has a portion that can slide parallel to the axis 212 (e.g., as directed by an operator's finger) or the control switch portion can be depressed. In some implementations, the control switch 240 can be moved to various positions to provide particular command signals (e.g., to select functions, options, or modes of the user interface system 120 and/or input device). In some implementations, one or more of the control switches 240 can be implemented as a button (e.g., depressed in a direction, such as perpendicular to the axis 212 or other direction), a rotary dial, a switch that moves perpendicular to the axis 212, or other type of input control. Control switches 240 can use electromagnetic sensors, mechanical switches, magnetic sensors, or other types of sensors to detect positions of the switch.

As previously stated, the computer-assisted system 100 can be a teleoperated system in which the user interface system 120 is, or is included in, a “leader” device that controls at least a portion of the manipulator assembly 130 (which in literature describing teleoperated systems may be known as a “follower” device).

In general, a control system 142 (e.g., a computing system) receives control signals from the user interface system 120 and generates actuation signals which are sent to manipulator assembly 130. The control system 142 can also receive sensor signals that indicate positions, orientations, states, and/or changes of various follower components (e.g., manipulator arm elements) from the manipulator assembly 130 and send actuation signals to the user interface system 120 to provide force, torque, and/or position feedback to the operator.

In one or more embodiments, a user interface system 120 is equipped with two controller portions 200 (one for each of the operator's hands) to control elements of the manipulator assembly 130 (e.g., instruments 160 attached to different manipulator arms 150A-D). An input device coordinate frame 201 is associated with the controller portions 200. In other words, the configuration (position and/or orientation) of the input device(s) 152 are determined within the input device coordinate frame 201 such that operations (e.g., kinematic operations) may be performed relative to a base frame of the manipulator assembly 130. For example, the position and/or orientation of an instrument 160 may be determined in the base frame using forward kinematics of the manipulator assembly 130 based on the configuration of one or more input devices 152.

While FIGS. 1-2B show various configurations of components, other configurations may be used without departing from the scope of the disclosure. For example, various components may be combined to create a single component. As another example, the functionality performed by a single component may be performed by two or more components. While examples of particular manipulator assemblies, particular repositionable structures, instruments, input systems, and controller portions are shown, the disclosure generalizes to any type of manipulator assemblies, repositionable structures, instruments, input systems, and controller portions with any number of degrees of freedom. Further, while components are often described in context of medical scenarios such as surgical scenarios, embodiments of the disclosure are equally applicable to other domains that involve robotic manipulation, e.g., non-surgical scenarios or systems, non-medical scenarios or systems, and/or the like.

FIGS. 2C-2D show examples of a manipulator assembly 130, in accordance with one or more embodiments.

In some embodiments, the manipulator assembly 130 introduces a set of instruments 260, 270, 280 (e.g., contained within one or more cannula) to a work site through one or more apertures (e.g., a port, an entry guide, an orifice). The manipulator assembly 130 includes separate manipulator arms 150A-C (e.g., a multi-port manipulator assembly 130, as shown in the inset of FIG. 2C) that support instruments 260, 270, 280, respectively. Movements of each of the instruments 260, 270, 280 may be independently controlled. Each of the instruments 260, 270, 280 supports an end effector 264, 274, 284 that may be positioned with one or more degrees of freedom (e.g., a 6 DOF instrument) by controlling the position and orientation of the joints and the links of the manipulator arm 150A-C that support instruments 260, 270, 280, respectively.

Instruments 260 and 270 are illustrated as equipped with end effectors 264, 274 that each include one or more degrees of freedom. For example, each end effector may be able to translate position in three dimensions or rotate orientation (e.g., pitch, yaw, roll). In case of a jawed end effector, an additional degrees of freedom may further enable and open/close operation of the jaws. As shown, instruments 260 and 270 may be two independently teleoperated instruments, each associated with a separate input device 152 (e.g., one left hand input device 152L for the left instrument 270 and one right hand input device 152R for the right instrument 260).

Instrument 280 is illustrated as equipped with an imaging device (e.g., an endoscopic camera) as the end effector 284. The imaging device may be any type of imaging sensor. The imaging device may be a general-purpose monoscopic or stereoscopic camera or it may be a specialized instrument (e.g., an endoscope). Additional details may be found in U.S. Pat. No. 8,620,473, (e.g., in FIG. 8 showing an articulable imaging system (1750) and the associated description).

The imaging device may be movable with one or more degrees of freedom. The imaging device may be able to articulate separately from other instruments 260, 270 that may be present in the workspace. The imaging device may have its own joints and links, in addition to the joints and links of the manipulator assembly 130. Any number of joints and/or links may be present, as long as the number of joints is sufficient to enable movement. More generally, the combination of the manipulator assembly 130 and the imaging device is not limited to a particular kinematic configuration (see alternative embodiment in FIG. 2D).

For example, the end effector 284 may be able to change orientation in pitch and yaw degrees of freedom (arrows G and H, respectively). In some embodiments, instrument 280 further includes a degree of freedom for rotation of the instrument shaft 282 (arrow I). The imaging device of instrument 280 may be used to observe operations performed using the end effectors 264, 274 of instruments 260, 270 by an operator viewing images captured from the end effector 284. If the operator wants to change the field of view of the imaging device, for example to view the operations performed using the end effectors 264, 274 from a different angle, the imaging device may be translated and/or rotated by control of instrument 280 (e.g., by an input device 152, by a programmed movement, by manual movement).

While the manipulator assembly 130 shown in FIG. 2C multiple manipulator arms 150 that separately support a plurality of instruments 260, 270, 280 and are integrated into a single manipulator assembly 130 (e.g., a multi-port manipulator assembly 130, as shown in the inset of FIG. 2C), other embodiments are also possible. For example, the manipulator assembly 130 may include one or more manipulator arms 150 on separate carts that may be independent controlled and that communicate with each other and or a user interface system 120.

Alternatively, FIG. 2D shows a manipulator assembly 130 that includes a manipulator-supporting link 250 that supports a plurality of instruments 260, 270, 280 (e.g., a single-port manipulator assembly 130, as shown in the inset of FIG. 2D). The manipulator-supporting link 250 may be rotated (arrow E) about insertion axis 290 of the manipulator-supporting link 250. Translational movement of each of the instruments 260, 270, 280 may be independently controlled (e.g., arrow F, for simplicity shows translation along the insertion axis for instrument 280). While FIG. 2D shows a jointed configuration of instrument shaft 282 that offsets the end effector 284 from instrument axis 283, the end effector 284 may be aligned with the instrument axis 283 (e.g., the instrument axis 283 of the imaging device is aligned with insertion axis 290).

As discussed above, any movement of the imaging device will alter the spatial relationship between the field of view and instruments in the workspace. Because the coordination of the operator (e.g., manipulating input device(s) 152) in the input device coordinate frame 201) relies on the operator's observation of the field of view, a mismatch (e.g., caused by a roll movement of the imaging device), would be undesirable.

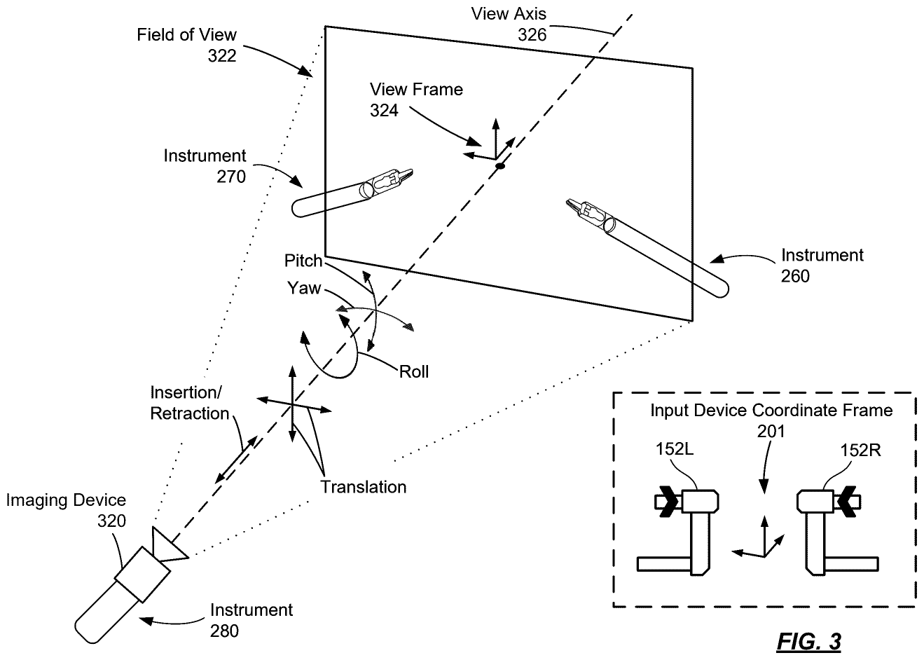

FIG. 3 is a simplified diagram showing example coordinate frames relevant to a computer-assisted system 100, in accordance with one or more embodiments.

In FIG. 3, the manipulator assembly 130 supports the instrument 280 equipped with an imaging device 320, the first instrument 260, and the second instrument 270. When operating the computer-assisted system 100 to perform a procedure, the operator may rely, at least partially, on visual feedback from the workspace. The workspace may include a target site, and the operator may, for example, want to visually inspect the target site and/or interact with the target site (e.g., by performing a procedure). The imaging device 320 may provide visual feedback based on a field of view 322 of the imaging device 320 (e.g., a 3D or 2D view of the field of view 322 determined by the optical characteristics of the imaging device 320).

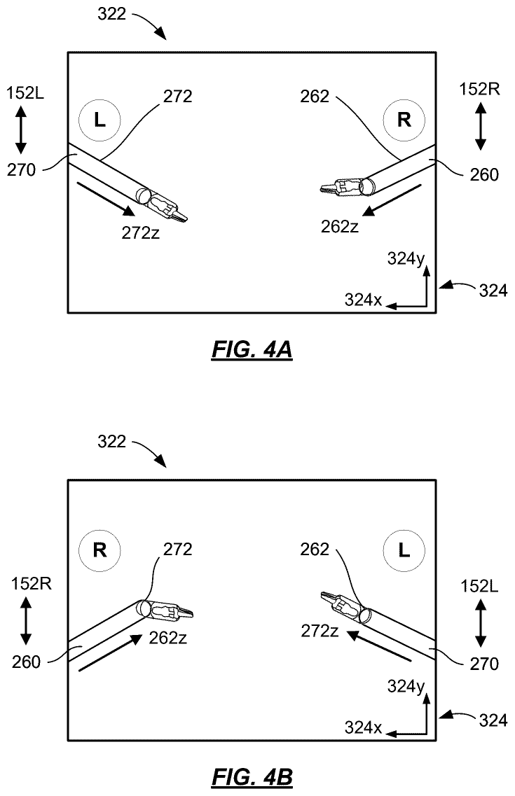

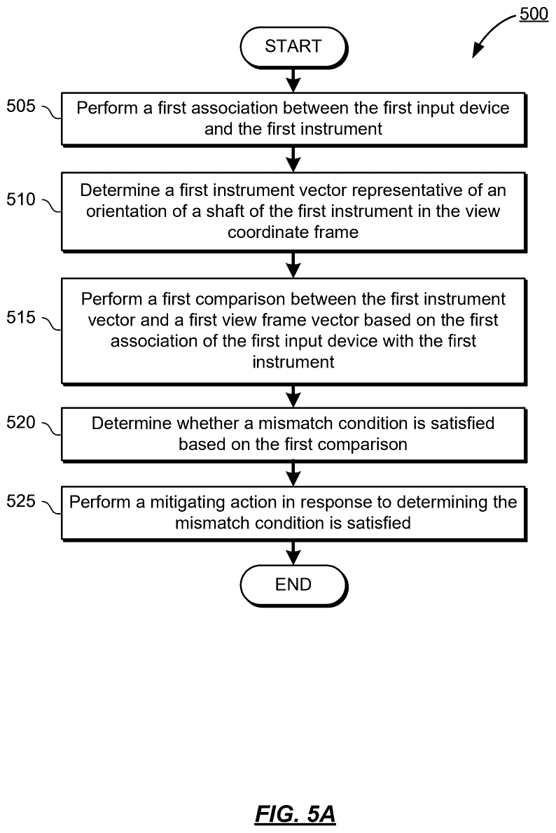

A view coordinate frame 324 is associated with the field of view 322. The view coordinate frame 324 is fixed relative to the field of view 322 (e.g., at the center of the field of view 322 as shown in FIG. 3, at a corner of the field of view as shown in FIGS. 4A-B). The view coordinate frame 324 is defined by a plurality of view frame vectors (e.g., orthogonal unit vectors, any appropriate coordinate vector system). In some embodiments, a first view frame vector defines a horizontal axis of the field of view 322, a second view frame vector defines a vertical axis of the field of view 322 and a third view frame vector is parallel to a view axis 326 that extends perpendicular to the field of view 322. It will be appreciated that other configurations of view frame vectors may be used to define the view coordinate frame 324.

The operator may want to change the field of view 322 for various reasons (e.g., to better observe the target site). While the field of view 322 may be modifiable using optical and digital operations (e.g., optical/digital zoom, digital translation and/or roll operations), in the following discussion, the field of view 322 is assumed to be changed by movement of the imaging device 320. The position and/or orientation of the imaging device 320 may be updated as desired by the operator, by changing the configuration of the joints of the manipulator assembly 130 (e.g., movement of manipulator assembly 130, manipulator arm 150, and/or instrument 280).

In some embodiments, one or more of the operating modes of the manipulator assembly 130 may enable the operator to control movement of the field of view 322 by moving an input device 152 (e.g., handle 202 of the controller portion 200 of the input device 152) which translates into commanded motion of the imaging device 320. Examples of robotic systems operating in different operating modes when receiving user inputs are provided in U.S. Pat. No. 9,586,323. U.S. Pat. No. 9,586,323 is hereby incorporated by reference in its entirety. The motion of the imaging device 320 may include one or more components (e.g., insertion/retraction/translation components, rotation components (e.g., a roll component, a pitch component, a yaw component)), as illustrated in FIG. 3.

In some embodiments, the configuration (position and/or orientation) of the imaging device 320 and therefore the field of view 322 may be directly manipulated by the operator (e.g., manual adjustment of imaging device 320, instrument 280, manipulator arm 150, manipulator assembly 130).

In general, the movement of the imaging device 320 and/or the field of view 322 may be summarized with a roll component and one or more non-roll components comprising the one or more translation components, the pitch component, the yaw component, and/or the insertion/retraction component. The motion of the field of view 322 may be described relative to any reference frame. In some embodiments, the motion of the field of view 322 may be described relative to the view coordinate frame 324 (e.g., a current view coordinate frame may be obtained relative to a previous view coordinate frame, or a future view coordinate frame may be obtained relative to a current view coordinate frame). In some embodiments, the motion of the field of view 322 may be described relative to a base of the manipulator assembly 130, which may be stationary or may be movable (e.g., installed on tracks enabling a linear motion, installed on another manipulator arm).

In FIG. 3, the instruments 260, 270 each support end effectors within the workspace being imaged by imaging device 320. In some embodiments, during a teleoperation control session, the movements of the instruments 260, 270 mimic the operator's manipulation of the input devices 152R, 152L, respectively, in the input device coordinate frame 201 (e.g., within the user interface system 120 of FIG. 2A). In this non-limiting example, the instrument 260 is manipulated based on input from input device 152R and the instrument 270 is manipulated based on input from input device 152L. It will be appreciated that the manipulator assembly 130 may be configured with any association between one or more input device(s) 152 and instrument(s) 160 in the workspace. For example, in some embodiments, control of any instrument may be switched between different input devices 152 or vice versa.

A high degree of coordination between a given input device 152 and the manipulator assembly 130 is required, especially during teleoperation control session (as known as a “following” mode) where the movement of an associated instrument 160 mimics the movement of the input device 152. If coordination between the movements of the input device 152 in the input device coordinate frame 201 the movements of the manipulator assembly 130 in the workspace is lost or degraded, operation of the computer-assisted system 100 may become non-intuitive to the operator. For example, lack of coordination may result in unintended movements of the associated instrument 160 (e.g., moving in the wrong direction). Therefore, one or more embodiments disclosed herein are directed to identifying and rectifying configurations of the computer-assisted system 100 that may result in non-intuitive motion.

As discussed above, in some embodiments, an imaging device 320 of the manipulator assembly 130 may be configured to move such that the operator can view the workspace from different perspectives. However, any movement of the imaging device 320 will alter the spatial relationship between the field of view 322 of the workspace and the input device coordinate frame 201. In other words, while the spatial relationship between the input device 152 and the associated instrument 160 may continue to operation within the teleoperation control session, the field of view 322 that the operator views in the display unit 154 may be offset (e.g., rotated) causing the perceived motion of the instrument 160 to not match the actual motion of the operators hands in the input device 152.

In some instances, the offset of the imaging device 320 may be caused if a commanded movement is different from the actual movement of the imaging device 320 (e.g., obstructed movement, back driving of a positioning motor). As a result of the incomplete movement command, the new field of view 322 may not match the expected spatial relationship and subsequent movement of the manipulator assembly 130 will become non-intuitive when viewed through the imaging device 320.

In some embodiments, the control system 142 monitors the relative orientation of one or more instruments 160 to identify a configuration that may cause non-intuitive motion. For example, the control system 142 may be configured to determine whether or not the orientation of an instrument 160 is reversed based on a comparison of an orientation vector of the instrument 160 relative to the view coordinate frame 322 of the imaging device 320.

FIGS. 4A-4B show example fields of view 322 of an imaging device 320, in accordance with one or more embodiments.

In FIG. 4A, the instrument 260 is manipulated based on input from input device 152R and the instrument 270 is manipulated based on input from input device 152L. Based on these assignments, a first association is made between the input device 152R and the instrument 260. Because the input device 152R is the right-side input of the user interface system 120, the first association indicates that the instrument 260 has an orientation that corresponds to the right-side of the field of view 322. Similarly, a second association may be made between the input device 152L (i.e., the left-side input of the user interface system 120) and the instrument 270, indicating that the instrument 260 has an orientation that corresponds to the left-side of the field of view 322.

In some embodiments, an association between an input device 152 and an instrument 160 may be represented by a quantitative equation. For example, in FIG. 4A-4B, the view coordinate frame 324 is defined such that a positive x-axis unit vector 324x extends from the right-side to the left-side of the field of view 322. Therefore, the first association (i.e., a right-side association of instrument 270) can be represented as requirement that a dot product of a first instrument vector 262z (i.e., the orientation of the shaft 262 of instrument 260) and unit vector 324x is positive (i.e., the shaft 262 is at least partially aligned with the unit vector 324x, indicating a right-to-left orientation or right-side association). Similarly, the second association (i.e., a left-side association of instrument 270) can be represented as requirement that a dot product of a second instrument vector 272z (i.e., the orientation of the shaft 272 of instrument 270) and unit vector 324x is negative (i.e., the shaft 272 is at least partially anti-aligned with the unit vector 324x, indicating a left-to-right orientation or left-side association).

While embodiments of this disclosure are described with respect to right-side association and left-side association, it will be appreciated that any directional association can be established between an input device 152 and an instrument 160. For example, any input device 152 (e.g., input device 152R) may be associated with any instrument 160 on any side of the field of view 322 (e.g., left, right, top, bottom) and the corresponding association may be quantified relative to any appropriate vector of the view coordinate frame 324 to describe the association.

A mismatch condition occurs when the comparison of an orientation vector of an instrument conflicts with the established association of the instrument. For example, in FIG. 4B, a mismatch condition is satisfied because the right-side association of instrument 260 is contradicted by the appearance of the instrument 260 on the left-side of the view of view 322. In this mismatch configuration, an upward motion of the first input device 152R in the input device coordinate frame 201 will cause the instrument 260 to move in a downward direction on the left-side of the field of view 322. While this example of non-intuitive motion is caused by a complete 180 degree rotation, other examples of non-intuitive motion can occur with any amount of rotation.

In some embodiments, determination of whether the mismatch condition is satisfied may be based on multiple associations. For example, in FIG. 4B, in addition to the mismatch condition related to instrument 260, the mismatch condition is also satisfied because the left-side association of instrument 270 is contradicted by the appearance of the instrument 270 on the right-side of the view of view 322. In this mismatch configuration, motion of both instruments 260, 270 will be non-intuitive (e.g., upward motion of the first input device 152R in the input device coordinate frame 201 will cause the instrument 260 to move in a downward direction on the left-side of the field of view 322, upward motion of the second input device 152L in the input device coordinate frame 201 will cause the instrument 270 to move in a downward direction on the right-side of the field of view 322). With more associations compromised by the mismatch condition, an operator may have considerable difficulty understanding the how the degrees of freedom for movement have been altered.

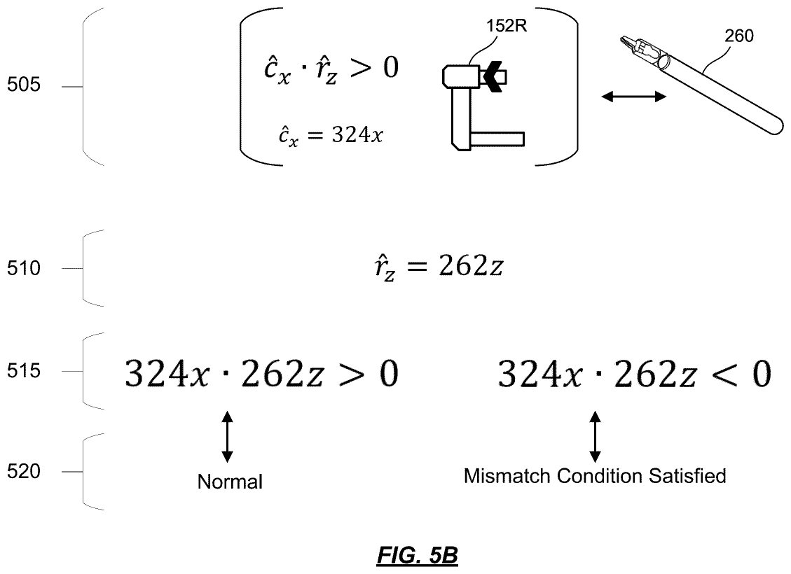

FIGS. 5A-5B show a method 500 according to one or more embodiments.

At 505, the control system 142 performs a first association between the first input device and the first instrument. In some embodiments, the first association may be recorded in a memory or storage device. As described above in the non-limiting example of FIG. 4A-4B, the first association is between the first input device 152R and the first instrument 260. The first association (i.e., a right-side association of instrument 260) may be mathematically represented by an equation including a dot product of a first instrument vector 2622 (i.e., the orientation of the shaft 262 of instrument 260) and a vector in the view coordinate frame 324. In this non-limiting example, because the instrument 260 is associated with a right-side input device 152R, the unit vector 324x, which points from right to left in the field of view 322 may be used. Accordingly, the dot product representation of the right-side association would have a positive value because the shaft 262, which enters the right-side of the field of view 322 for the right-side association, is at least partially aligned with the unit vector 324x.

In other words, the first association may be a heuristic rule that describes an expected relationship between an input device 152 and an instrument 160. For example, the above dot product of unit vector 324x and the shaft 262z is expected to have a positive value for a right-side association (positive/negative value is sufficient and the magnitude of the dot product is not used). While this description describes one or more embodiments based on the sign of a dot product (i.e., alignment or anti-alignment of an instrument shaft with a vector selected based with the input device), other representations of the first association may be used (e.g., other vector selections based on the input device, other mathematical representations).

At 510, the control system 142 determines the first instrument vector 262z that represents the orientation of the shaft 262 of the first instrument 260 in the view coordinate frame 324. In some embodiments, the control system 142 is configured to determine the first instrument vector 262z using kinematic information of the first instrument 260. For example, the control system 142 may use defined kinematic relationships between links and joints of the manipulator assembly 130 (e.g., Denavit-Hartenberg (DH) parameters) to determine an absolute orientation (e.g., relative to a world frame) of the first instrument 260 and the instrument 280 supporting the imaging device 320 or relative orientation (e.g., instrument orientation relative to view coordinate frame 324).

In some embodiments, the first instrument vector is determined utilizing a computer vision technique to identify the shaft 262 of the first instrument 260 in the field of view 322 (i.e., in an image generated by the imaging device 320).

At 515, the control system 142 performs a first comparison between the first instrument vector and a first view frame vector based on the first association of the first input device with the first instrument. As discussed above, the first association (i.e., a right-side association of instrument 260) may be mathematically represented by a dot product that is expected to have a positive value (i.e., unit vector 324x and the shaft 262 are expected to be at least partially aligned in the right-side association of instrument 260). Accordingly, based on this first association of the first input device 152R with the first instrument 260, the first comparison may include performing an actual calculation of the dot product using the determined first instrument vector 262z and actual unit vector 324x based on the orientation of the imaging device 320. It will be appreciated that when the first association is described using a different heuristic or mathematical rule, the first comparison would include an appropriate calculation (with the first instrument vector 262z) that is based on the corresponding first association. For example, in other embodiments, any appropriate vector of the view coordinate frame 324 that relates to the first association (e.g., vector corresponding to the horizontal axis, vertical axis, user defined axis of the field of view) may be used in a dot product, cross product, etc.

At 520, the control system 142 determines whether a mismatch condition is satisfied based on the first comparison.

Following the non-limiting example of FIG. 4A-4B, the first association dictates that the above dot product of unit vector 324x and the shaft 262z is expected to have a positive value (i.e., the right-side association of the instrument 260). Therefore, the mismatch condition is satisfied if the dot product calculated in the comparison from 515 has a negative value that contradicts the first association. In other words, the calculated dot product of the first instrument vector 262z and the first view frame vector 324x has an opposite sign relative to the first association.

However, the mismatch condition is not limited to an exact opposite relationship to the first association. For example, if the above dot product is zero (i.e., the instrument is perpendicular to the first view frame vector 324x) or less than a threshold, the mismatch condition may be satisfied. In this non-limiting example, a dot product with a near-zero magnitude means the instrument 260 is nearly vertical in the field of view 322. Because the vertical orientation does not have a clearly associated with a right or left side of the field of view, there is a potential for a non-intuitive control scheme and the mismatch condition may be satisfied.

In some embodiments, the control system 142 is configured to determine whether the mismatch condition is satisfied in response to the operator enabling a teleoperational control session of the manipulator assembly 130. In other words, when the instrument 260 is expected to “follow” motion of the input device 152R, the control system 142 determines whether the mismatch condition is satisfied to prevent unintended motion or a non-intuitive control scheme.

At 525, in response to determining that the mismatch condition is satisfied, the control system 142 performs a mitigation action.

In some embodiments, a mitigating action may include a prompt or appropriate indicator (e.g., visual, audible, tactile, etc.) directed to the operator. In certain embodiments, a user interface element (e.g., a banner message) regarding the mismatch condition may be displayed within the field of view displayed to the operator of the computer-assisted system 100. In addition, user interface elements regarding the mismatch condition may be displayed on one or more other displays (e.g., display unit 154, an external display) of the computer-assisted system 100 (e.g., on a tower display for personnel within an environment of the manipulator assembly 130, on a second input system (e.g., a dual console) communicatively coupled to the manipulator assembly 130, etc.).

In some embodiments, the mitigating action includes an instruction (e.g., conveyed using text and/or images and/or audio) for the operator to remove and reinstall the imaging device 320 in the manipulator assembly 130. The mitigating action may further include an instruction to replace the image device 320. For example, in certain embodiments, the computer-assisted system 100 may be configured to determine, based on system log data, that a particular imaging device 320 has caused multiple mismatch condition events (e.g., multiple mismatch condition events within a certain time period, multiple consecutive mismatch condition events, etc.). This may indicate a hardware malfunction on the imaging device 320. In response, the computer-assisted system 100 may be configured to display an instruction (e.g., displayed on the input system within the field of view of the operator and/or displayed on a tower display to personnel within an environment of the manipulator assembly, etc.) to replace the faulty imaging device 320. Furthermore, in some embodiments, upon determining that the mismatch condition exists, the control system 142 may be configured to prevent movement of the first instrument 260 until the imaging device 320 is reinstalled or replaced. Reinstalling or replacing the imaging device 320 may cause the control system 142 to recalibrate the associations between the input device(s) 152 and the instrument(s) 160 (i.e., reassociate the input device(s) 152 and the instrument(s) 160).

In some embodiments, the mitigating action includes preventing, by the control system 142, movement of the manipulator assembly 130. For example, movement of one or more instrument(s) 160 may be prevented or at least partially restricted. In some cases, the movement may be prevented until the mismatch condition is resolved (e.g., by reinstalling the imaging device 320, by reassociating the input device 152).

In some embodiments, the mitigation action includes pausing or terminating any teleoperational control session based on the mismatch condition being satisfied (e.g., to immediately prevent unintended motion). For example, an operational mode of the computer-assisted system 100 may be changed to alter the behavior of the manipulator assembly 130.

In some embodiments, the mitigating action includes reassociating one or more input device(s) 152 and one or more instrument(s) 160. For example, an association between the input device 152 and the corresponding instrument 160 may be redefined based on the current configuration of the instrument 160. In some cases, where multiple input devices 152 and multiple instrument(s) 160 are configured, the reassociation may change the mapping between the input devices 152 and the multiple instrument(s) 160. For example in the configuration shown in FIG. 4B, the mitigating action may include reassociating the input device 152R with the second instrument 270 and reassociating the second input device 152L with the first instrument 260.

While FIGS. 5A-5B are described in the context of the right-side association of instrument 260, the first association may alternatively be based on a left-side association of the instrument 270 (i.e., with the left input device 152L), as summarized below with an “*” notation.

At 505*, the first association may be represented by a dot product of unit vector 324x and the shaft 272z with a negative value for a left-side association (i.e., the shaft 272, which enters the left-side of the field of view 322 for the left-side association, is at least partially anti-aligned with the unit vector 324x).

At 510*, the control system 142 determines the first instrument vector 272z that represents the orientation of the shaft 272 of the first instrument 270 in the view coordinate frame 324.

At 515*, based on this first association of the input device 152L with the first instrument 270, the first comparison may include performing an actual calculation of the dot product using the determined first instrument vector 272z and actual unit vector 324x based on the orientation of the imaging device 320.

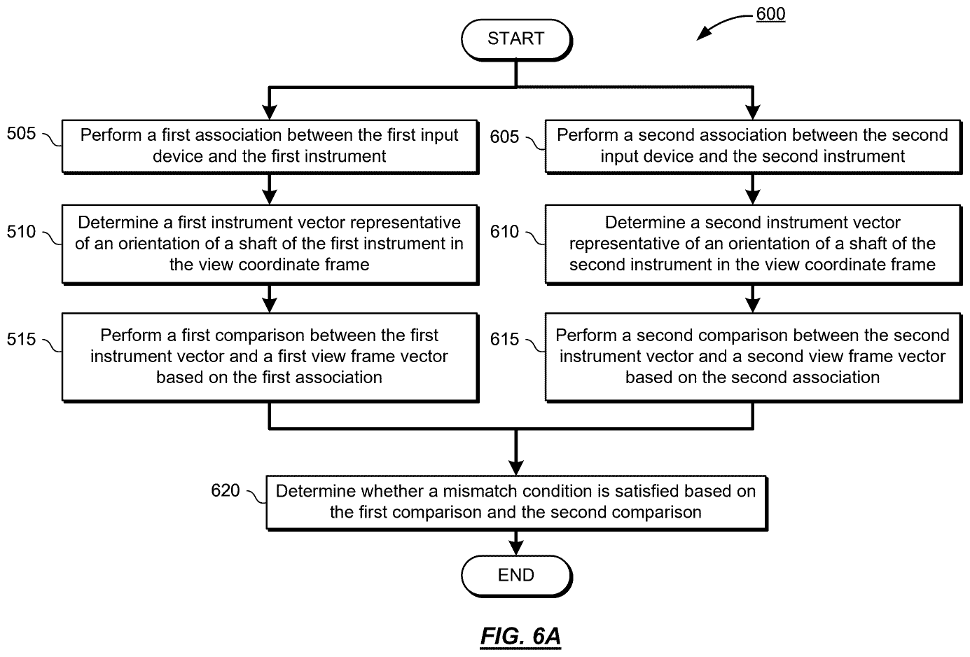

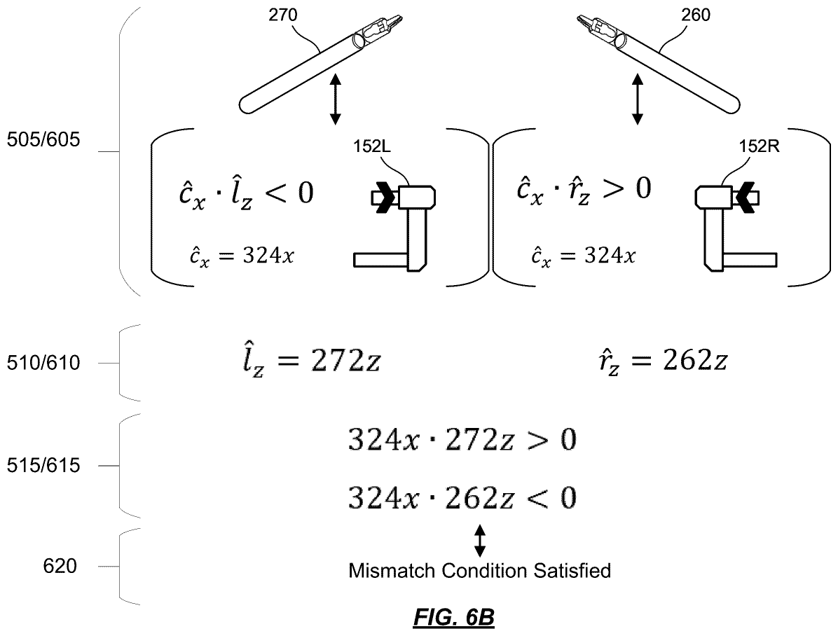

At 520*, the control system 142 determines whether a mismatch condition is satisfied based on the first comparison. Because the dot product of first instrument vector 272z and actual unit vector 324x in FIG. 4B would be positive (i.e., the instrument 270 extends from the right side in FIG. 4B), the first association from 505* is contradicted and the mismatch condition is satisfied. FIGS. 6A-6B show a method 600 according to one or more embodiments.

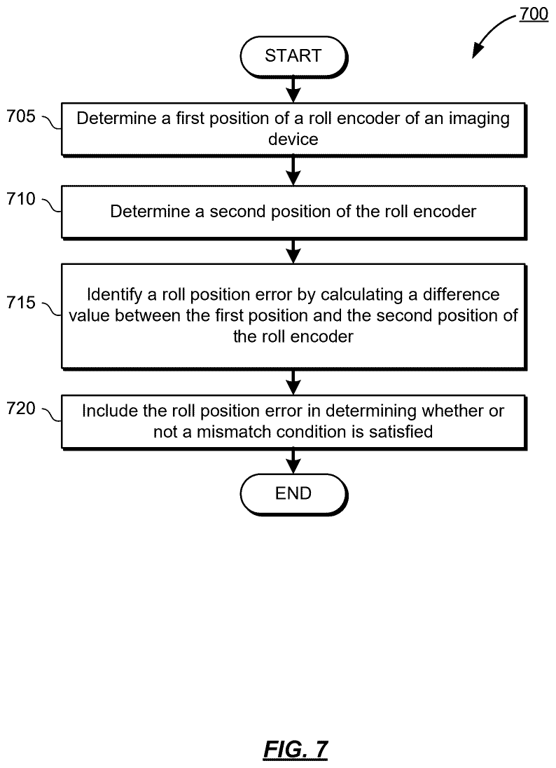

In some embodiments, a determination of whether the mismatch condition is satisfied may be based on multiple associations. For example, when a manipulator assembly 130 is equipped with instruments 260, 270 and an imaging device 320, there is a possibility that the 260, 270 instruments appear swapped in the field of view 322 (see FIG. 4B). As discussed in further detail below with respect to FIG. 7, this scenario may be caused by movement or rotation of the imaging device 320 within the workspace.

At 605, the control system 142 performs a second association between the second input device and the second instrument. In some embodiments, the second association may be recorded in a memory or storage device. As described above in the non-limiting example of FIG. 4A-4B, the second association is between the second input device 152L and the second instrument 270. The second association (i.e., a left-side association of instrument 270) may be mathematically represented by an equation including a dot product of a second instrument vector 272z (i.e., the orientation of the shaft 272 of instrument 270) and unit vector 324x. The dot product representation of the left-side association would have a negative value because the shaft 272, which enters the left-side of the field of view 322 for the left-side association, is at least partially anti-aligned with the unit vector 324x. While this non-limiting example of the second association uses the same unit vector 324x as the first association, in some embodiments, the first association and the second association may not directly correspond and/or may be unrelated from each other (i.e., use entirely different representations or metrics for comparisons).

At 610, the control system 142 determines the second instrument vector 272z that represents the orientation of the shaft 272 of the second instrument 270 in the view coordinate frame 324. In some embodiments, the control system 142 is configured to determine the second instrument vector 272z using kinematic information of the second instrument 270. For example, the control system 142 may use defined kinematic relationships between links and joints of the manipulator assembly 130 (e.g., Denavit-Hartenberg (DH) parameters) to determine an absolute orientation (e.g., relative to a world frame) of the second instrument 270 and the instrument 280 supporting the imaging device 320 or relative orientation (e.g., instrument orientation relative to view coordinate frame 324).

In some embodiments, the second instrument vector 262z is determined utilizing a computer vision technique to identify the shaft 272 of the second instrument 270 in the field of view 322 (i.e., in an image generated by the imaging device 320).

At 615, the control system 142 performs a second comparison between the second instrument vector and a second view frame vector based on the second association of the second input device with the second instrument. As discussed above, the first association (i.e., a right-side association of instrument 260) may be mathematically represented by a dot product that is expected to have a positive value (i.e., unit vector 324x and the shaft 262 are expected to be at least partially aligned in the right-side association of instrument 260). Accordingly, based on this first association of the first input device 152R with the first instrument 260, the second comparison may include performing an actual calculation of the dot product of the second instrument vector 272z and actual unit vector 324x based on the orientation of the imaging device 320. It will be appreciated that when the first association is described using a different heuristic or mathematical rule, the second comparison would include an appropriate calculation (with the second instrument vector 272z) that is based on the corresponding second association. For example, in other embodiments, any appropriate vector of the view coordinate frame 324 that relates to the second association may be used in a dot product, cross product, etc.

At 620, the control system 142 determines whether a mismatch condition is satisfied based on the first comparison and the second comparison. The determination may require that one comparison indicates a mismatch condition. Alternatively, the determination may require that both comparisons indicate a mismatch condition. As shown in the non-limiting example of FIG. 6B, the mismatch condition requires that: in the first comparison, a dot product of the first instrument vector and the view frame vector has an opposite sign relative to the first association; and in the second comparison, a dot product of the second instrument vector and the view frame vector has an opposite sign relative to the second association.

FIG. 7 shows a method according to one or more embodiments.

In some embodiments, the manipulator assembly may be configured with features that relate to determining whether a mismatch condition exists. Accordingly, the control system may be configured to monitor for a mismatch condition during use of these features. For example, the mismatch condition may have an additional requirement that a feature is currently in use. Alternatively, or in addition, the mismatch condition may have an addition requirement related to one or more parameters that are specific to the feature of the manipulator assembly.

For example, an imaging device 320 of the manipulator assembly 130 may be configured with a roll axis of the imaging device 320 (e.g., rotation I about instrument axis 283 in FIG. 2D, rotation G/H of a joint in the instrument 280 that supports the imaging device 320 in FIG. 2C). Because movement, particularly rotation, of the imaging device 320 may alter the spatial relationship between the field of view 322 and instruments in the workspace, the control system 142 may be configured to monitor for a mismatch condition based on a roll movement.

At 705, the control system 142 determines a first position of a roll encoder of an imaging device 320. The roll encoder may be a sensor (e.g., potentiometer), within the instrument 280 supporting the imaging device 320, that identifies a position of the imaging device 320. In some embodiments, the roll encoder is a processor that determines a position of the imaging device 320 based on kinematic information of the manipulator assembly 130 and/or the instrument 280.

At 710, the control system 142 determines a second position of the roll encoder.

In some embodiments, the second position may be different from the first position because the control system 142 performs a roll movement of a given rotation angle about an axis. The roll movement may be performed in combination with other movements of the manipulator assembly 130. The given rotation angle and the axis may be determined based on any appropriate source (e.g., input by an operator, predetermined by a procedure or subroutine).

In some embodiments, the second position may be different from the first position because of a movement that is not accounted for by the control system 142. For example, a motor or servo mechanism of the manipulator may be back driven, an operator may directly manipulate the instrument (e.g., manual repositioning). In some cases a commanded roll movement may be interrupted or otherwise incomplete (e.g., obstruction in the workspace, resistance, such as tension on cables or wires of the manipulator assembly 130), causing a portion of the commanded roll movement to be unaccounted for by the control system 142.

At 715, the control system 142 identifies a roll position error by calculating a difference between positions of the roll encoder (e.g., the difference between the second position the first position). The difference may be caused by interrupted movement (e.g., collision, resistance), back driving of a motor or servomechanism, operator manipulation of the instrument (e.g., manual repositioning), any movement of the manipulator assembly 130, or any combination thereof.

At 720, the control system 142 includes the roll position error in determining whether or not a mismatch condition is satisfied. In some embodiments, the mismatch condition includes an error requirement that the roll position error is greater than a threshold.

In some embodiments, the determination of whether a mismatch condition is satisfied may be terminated under one or more conditions (e.g., a commanded roll movement is smaller than a threshold, the roll axis is excluded from the mismatch condition, the roll position error is smaller than a threshold).

In some embodiments, method 700 relates to a manipulator assembly 130 configured to perform a camera roll of 180 degrees about an axis (e.g., a “scope flip” including rotation I about view axis 326 in FIG. 2D). While the camera roll may provide the operator with a convenient new perspective of the workspace, any movement that deviates from the predetermined angle of 180 degrees may result in non-intuitive motion. For example, if the imaging device 320 fails to move at all during the camera roll, the orientation of one or more instruments may be reversed in the field of view 322 (see FIG. 4B).

In some embodiments, the control system 142 is configured to determine whether the mismatch condition is satisfied during the camera roll. In other words, because any movement of the imaging device 320 (e.g., the camera) may result in non-intuitive motion due the change in the field of view 322, the mismatch condition may have multiple requirements based on the camera roll feature. For example, in addition requiring (A) that the first comparison fails to satisfy the first association, the mismatch condition may additionally require (B) that the first comparison is performed during or as a result of the camera roll. In some embodiments, the mismatch condition may further require (C) that a roll position error during the camera roll is greater than a threshold. In some embodiments, the mismatch condition may further require (D) that one or more other requirements are satisfied when and/or after the operator enables a teleoperational control session. It will be appreciated that the mismatch condition may include any or all of requirements (A), (B), (C), and (D) described above. Furthermore, the mismatch condition may further include additional requirements within the scope of this description.

Although methods 500, 600, 700 have been described with respect to a limited number of examples and operations, those skilled in the art, having benefit of this disclosure, will appreciate that various other embodiments may be devised without departing from the scope of the present disclosure.

Furthermore, while the various blocks in the flowcharts are presented and described sequentially, one of ordinary skill in the art will appreciate that some or all of the blocks may be executed in different orders, combined, omitted, and some or all of the blocks may be executed in parallel.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A computer-assisted system comprising:
a first input device configured to be manipulated by an operator;
a manipulator assembly configured to support an imaging device and a first instrument, wherein the imaging device has a field of view associated with a view coordinate frame; and
a control system communicatively coupled to the first input device and the manipulator assembly, wherein the control system is configured to:
perform a first association between the first input device and the first instrument;
determine a first instrument vector representative of an orientation of a shaft of the first instrument in the view coordinate frame;
perform a first comparison between the first instrument vector and a first view frame vector, the first comparison being performed based on the first association of the first input device with the first instrument;
based on the first comparison, determine whether a mismatch condition is satisfied; and
in response to determining that the mismatch condition is satisfied, perform a mitigation action.

2. The computer-assisted system of claim 1, further comprising:
a second input device configured to be manipulated by the operator,
wherein the manipulator assembly is configured to support a second instrument,
wherein the control system is configured to:
perform a second association between the second input device and the second instrument;
determine a second instrument vector representative of an orientation of a shaft of the second instrument in the view coordinate frame;
perform a second comparison between the second instrument vector and a second view frame vector, the second comparison being performed based on the
second association of the second input device with the second instrument; and
wherein the determination of whether the mismatch condition is satisfied is further based on the second comparison.

3. The computer-assisted system of claim 2,
wherein the first comparison comprises a dot product of the first instrument vector and the view frame vector, and
wherein the second comparison comprises a dot product of the second instrument vector and the view frame vector.

4. The computer-assisted system of claim 1,
wherein the mitigating action includes an instruction to remove and reinstall the imaging device in the manipulator assembly.

5. The computer-assisted system of claim 4,
wherein the mitigating action includes preventing, by the control system, movement of the first instrument until the imaging device is reinstalled.

6. The computer-assisted system of claim 1,
wherein the mitigating action includes preventing, by the control system, movement of manipulator assembly.

7. The computer-assisted system of claim 1,
wherein the mitigating action includes reassociating the first input device with the first instrument.

8. The computer-assisted system of claim 2,
wherein the mitigating action includes reassociating the first input device with the second instrument and reassociating the second input device with the first instrument.

9. The computer-assisted system of claim 1,
wherein the control system is configured to identify a roll position error by calculating a difference between positions of a roll encoder of the imaging device.

10. The computer-assisted system of claim 9,
wherein the mismatch condition includes an error requirement that the roll position error is greater than a threshold.

11. The computer-assisted system of claim 1,
wherein the control system is configured to determine whether the mismatch condition is satisfied in response to the operator enabling a teleoperational control session of the manipulator assembly.

12. The computer-assisted system of claim 11,
wherein the mitigating action includes terminating the teleoperational control session.

13. The computer-assisted system of claim 1,
wherein the imaging device is configured to perform a camera roll of 180 degrees about an axis.

14. The computer-assisted system of claim 13,
wherein the control system is configured to determine whether the mismatch condition is satisfied during the camera roll.

15. The computer-assisted system of claim 13,
wherein the control system is configured to determine whether the mismatch condition remains satisfied after the operator enables a teleoperational control session of the manipulator assembly.

16. The computer-assisted system of claim 13,
wherein the control system is configured to identify a roll position error by comparing positions of a roll encoder of the imaging device before and after the camera roll, and
wherein the mismatch condition includes an error requirement that the roll position error is greater than a threshold.

17. The computer-assisted system of claim 1,
wherein the control system is configured to determine the first instrument vector utilizing a computer vision technique to identify the shaft of the first instrument in an image generated by the imaging device.

18. The computer-assisted system of claim 1,
wherein the control system is configured to determine the first instrument vector using kinematic information of the first instrument.

19. A method of operating a computer-assisted system including a first input device configured to be manipulated by an operator, a manipulator assembly configured to support an imaging device and a first instrument, and a control system, the method comprising:
performing, by the control system, a first association between the first input device and the first instrument;
determining, by the control system, a first instrument vector representative of an orientation of a shaft of the first instrument in a view coordinate frame associated with a field of view of the imaging device;
performing, by the control system, a first comparison between the first instrument vector and a first view frame vector, the first comparison being performed based on the first association of the first input device with the first instrument;
determining, by the control system and based on the first comparison, whether a mismatch condition is satisfied; and
performing, by the control system and in response to determining that the mismatch condition is satisfied, a mitigation action.

20. A non-transitory machine-readable medium comprising a plurality of machine-readable instructions executed by one or more processors associated with a computer-assisted system including a first input device configured to be manipulated by an operator, a manipulator assembly configured to support an imaging device and a first instrument, and a control system, the plurality of machine-readable instructions causing the one or more processors to:
perform, by the control system, a first association between the first input device and the first instrument;
determine, by the control system, a first instrument vector representative of an orientation of a shaft of the first instrument in a view coordinate frame associated with a field of view of the imaging device;
perform, by the control system, a first comparison between the first instrument vector and a first view frame vector, the first comparison being performed based on the first association of the first input device with the first instrument;
determine, by the control system and based on the first comparison, whether a mismatch condition is satisfied; and
perform, by the control system and in response to determining that the mismatch condition is satisfied, a mitigation action.
